(19) United States
(12) Patent Application Publication
(10) Pub. No.: US 2025/0268621 A1
(43) Pub. Date: Aug. 28, 2025

(54) VARIABLE NAVIGATED EXPANDABLE SCALPEL DEVICE

(71) Applicant: GLOBUS MEDICAL, INC., AUDUBON, PA (US)

(72) Inventors: Corbett McLaughlin, Philadelphia, PA (US); Myles Sullivan, Philadelphia, PA (US); Carly Taubenkraut, Perkasie, PA (US); Mark Weiman, Downingtown, PA (US)

(21) Appl. No.: 18/589,712

(22) Filed: Feb. 28, 2024

(51) Int. Cl. A61B 17/3213; A61B 17/32; A61B 17/3211; A61B 34/30

(52) U.S. Cl. CPC A61B 17/3213; A61B 34/30; A61B 2017/320052; A61B 2017/32113

(57) ABSTRACT

Various implementations include a scalpel device and related surgical robots. In one implementation, a scalpel device includes: a body sized to translate within a guide tube for delivery to a patient; an expandable cutting tool coupled with a distal end of the body, the expandable cutting tool including a cutting assembly; and an actuator including a control feature coupled with a proximal end of the body, wherein the actuator enables adjustment of the cutting assembly according to an incision width selected from a set of incision widths defined by the control feature.

### TECHNICAL FIELD

This disclosure generally relates to the field of surgery and surgical devices. More particularly, the disclosure relates to scalpel devices and related apparatuses and systems.

### BACKGROUND

Surgical procedures such as spinal procedures vary based on a multitude of pathologies, treatment options, levels of complexity, and degrees of invasiveness. A non-limiting group of conditions that may require a surgery such as a spinal procedure can include degenerative disc disease, spinal stenosis, or spondylolisthesis. These conditions can require varying sizes of incisions and degrees of disruption to the local muscle, ligaments, and soft tissue in the process of gaining access to the location of surgery (e.g., spinal elements). Incision size and trajectory are conventionally created using radiographic imaging to mark locations superficially on the patient, which are later referenced when using a scalpel to create the incision. Cut sizes can vary based on surgeon preference, visual markings, and/or procedure. Further, surgeon variation can introduce a margin of error that can exacerbate iatrogenic effects related to initial access.

### SUMMARY

The needs above, as well as others, are addressed by embodiments of apparatuses for providing feedback on implants, as well as systems for providing implant feedback, and related methods described in this disclosure. All examples and features mentioned below can be combined in any technically possible way.

Various implementations include scalpel devices and related surgical robotic systems. Particular implementations include scalpel devices with an expandable cutting tool and an actuator that enables adjustment of the expandable cutting tool according to a selected incision width.

In particular aspects, a scalpel device includes: a body sized to translate within a guide tube for delivery to a patient; an expandable cutting tool coupled with a distal end of the body, the expandable cutting tool including a cutting assembly; and an actuator including a control feature coupled with a proximal end of the body, where the actuator enables adjustment of the cutting assembly according to an incision width selected from a set of incision widths defined by the control feature.

In further particular aspects, a surgical robot system includes the scalpel device. In certain cases, the surgical robot system further includes a robot having a robot base, a robot arm coupled to the robot base, and an end-effector coupled to the robot arm, the end effector including the guide tube for receiving the body of the scalpel device.

Implementations may include one of the following features, or any combination thereof.

In certain examples, the control feature includes a lock for fixing a position of the cutting assembly.

In particular cases, the cutting assembly includes a scalpel blade, and adjustment of the scalpel blade includes at least one of: changing an angle of the scalpel blade relative to an axis of the body, translating the scalpel blade from the body, or retracting the scalpel blade within the body.

In some implementations, the scalpel blade includes a plurality of scalpel blades pivotably coupled to one another.

In certain examples, the guide tube includes a dilator.

In particular aspects, the body includes an outer shaft and an inner shaft within the outer shaft.

In some cases, the outer shaft includes a set of external grooves for aligning the scalpel device with the guide tube, wherein the set of external grooves aids in orienting the cutting assembly relative to the patient.

In certain aspects, the actuator is coupled with a proximal end of the body.

In some implementations, the expandable cutting tool includes a cartridge that is removably coupled with a distal end of the body, where the cartridge includes: a main body, a set of blade mounts, and a set of blades connected with the set of blade mounts.

In particular cases, each blade has a cutting edge and a coupler for connecting with the set of blade mounts.

In some aspects, the expandable cutting tool includes a set of blade mounts coupled with a distal end of the body, where the set of blade mounts enable detachable coupling of at least one blade to the body.

In particular implementations, the actuator includes at least one of a push-to-actuate or a rotate-to-actuate feature.

In certain cases, the control feature includes a limiter that limits the push-to-actuate or rotate-to-actuate feature.

In some aspects, the limiter includes a release mechanism for releasing the actuator.

In particular cases, actuating the release mechanism causes the actuator to revert to a default position.

In some implementations, the actuator includes a visual indicator of the incision width.

In certain aspects, adjustment of the actuator causes a change in the visual indicator of the set of incision widths.

In particular implementations, the expandable cutting tool is sized to fit within the guide body when retracted and expand radially beyond the guide body when extended.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 shows a perspective view of a scalpel device and related system according to various implementations.

FIG. 2 illustrates actuation of the scalpel device from FIG. 1.

FIG. 3 shows a perspective view of a scalpel device according to various implementations.

FIG. 4 shows a perspective view of a scalpel device with a navigation device according to various implementations.

FIGS. 5A and 5B show perspective views of an outer housing in a scalpel device according to various implementations.

FIGS. 6A and 6B show perspective views of an inner housing in a scalpel device according to various implementations.

FIGS. 7A and 7B show perspective views of an actuator and cutting assembly in a scalpel device, in distinct positions, according to various implementations.

FIGS. 8A and 8B show perspective views of portions of a cutting assembly according to various implementations.

FIGS. 9A and 9B show perspective and side views, respectively, of a cutting assembly according to various implementations.

FIG. 10A shows a cross-sectional view of a mounting configuration for a cutting assembly according to various implementations.

FIG. 10B shows a perspective view of an alternative cutting assembly mounting configuration according to various implementations.

FIGS. 11A and 11B show perspective views of a blade mounting configuration according to various implementations.

FIGS. 12A-12C show perspective views of a blade mounting configuration according to various implementations.

FIGS. 13A and 13B show perspective views of a blade mounting configuration according to various implementations.

FIG. 14 is a perspective view of a scalpel device according to various additional implementations.

FIGS. 15A and 15B show perspective views of an actuator and corresponding cutting assembly, respectively, according to various implementations.

FIGS. 16A and 16B show perspective views of an actuator and corresponding cutting assembly, respectively, according to various implementations.

FIGS. 17A and 17B are side views of a cutting assembly in distinct positions according to various implementations.

FIGS. 18A-18D are side views of a cutting assembly in distinct positions according to various implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Various example embodiments of scalpel devices, surgical robotic systems, and approaches for using such devices and systems are described herein. In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The apparatuses and related systems and methods described herein boast a variety of inventive features and components that warrant patent protection, both individually and in combination.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

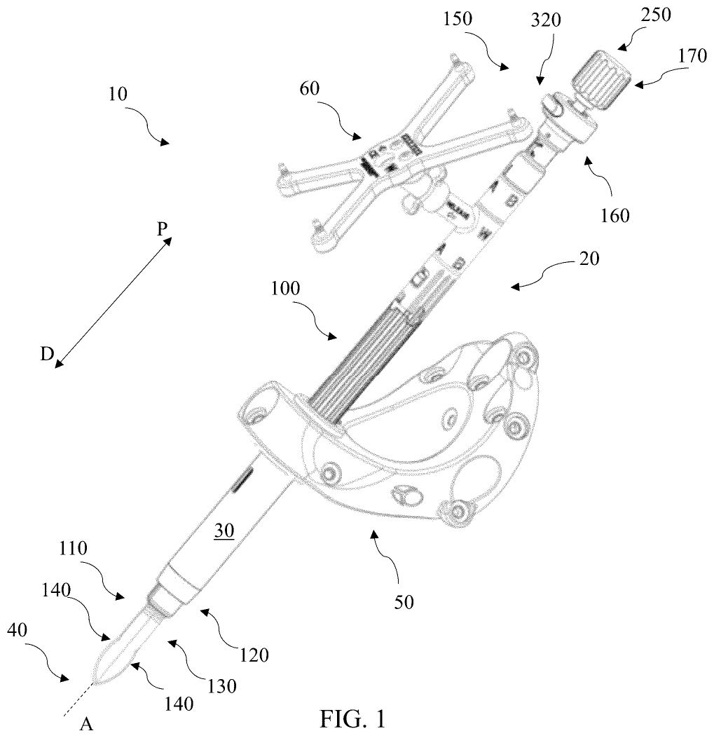

As disclosed herein, use of the term proximal refers to the direction away from attachment of an element to the subject, shown in FIG. 1 as direction P, while use of the term distal refers to the direction opposite the proximal direction and toward attachment of an element to the subject, shown in FIG. 1 as direction D. Commonly labeled components in the FIGURES are considered to be substantially equivalent components for the purposes of illustration, and redundant discussion of those components is omitted for clarity.

As noted herein, surgical incision size and trajectory are conventionally created using radiographic imaging to mark locations superficially on the patient, which are later referenced when using a scalpel to create the incision. Cut sizes can vary based on surgeon preference, visual markings, and/or procedure. Further, surgeon variation can introduce a margin of error that can exacerbate iatrogenic effects related to initial access.

Certain conventional approaches and instrumentation are described in U.S. Pat. No. 11,045,267 (Surgical Robotic Automation with Tracking Markers, issued Jun. 29, 2021), the entire contents of which are hereby incorporated by reference. In one conventional effort to streamline the incision making process, robotic procedures use a static scalpel blade fit to the end effector's inner diameter with the blade edge off-axis of centerline. The cutting technique involves an initial plunge through the end effector, then a 180 degree flip of the blade followed by a second plunge to complete the incision, creating a length of cut equal to the diameter of the end effector. This can result in the tubular instrumentation that follows being unable to fit and is a direct result of an insufficient cutting window and skin elasticity. When an operator attempts to address this issue, e.g., by creating an incision size greater than what this technique yields, such an attempt is accomplished outside the tube of the end effector and reintroduces human variability and error. Traditional practices do not incorporate navigation or robotic guidance into this step of surgery, which can ultimately lead to an increased and unnecessary amount of soft tissue disruption.

This disclosure provides, at least in part, a scalpel device that enables an operator such as a surgeon to control and select an incision width of a cutting assembly from a set of incision widths, for example, using an actuator at a proximal end of the device body. In certain cases, the scalpel enables fixing the position of the cutting assembly. In various implementations, the cutting assembly enables adjustment of an angle of a scalpel blade relative to the body, translation of the scalpel blade from the body, and/or retraction of the scalpel blade within the body.

FIG. 1 illustrates a system 10, which can include a robotic surgical system in various implementations. The system 10 includes a scalpel device 20 according to various implementations. In certain cases, the system 10 includes a guide tube 30 such as an end effector or a dilator that enables the scalpel device 20 to be loaded axially, e.g., through a hollowed out body of the guide tube 30 to reach a cutting location 40 such as an area proximate a patient's skin. In certain cases, an end effector 50 can be used to retain the guide tube 30. In particular examples, the end effector 50 is part of, or connected with, a robotic arm, such as in a robotic surgical system. Further, the system 10 can include an additional navigator 60 that can be coupled with the scalpel device 20, e.g., to enable positioning of the scalpel device 20. As noted herein, in various implementations the navigator 60 is optional.

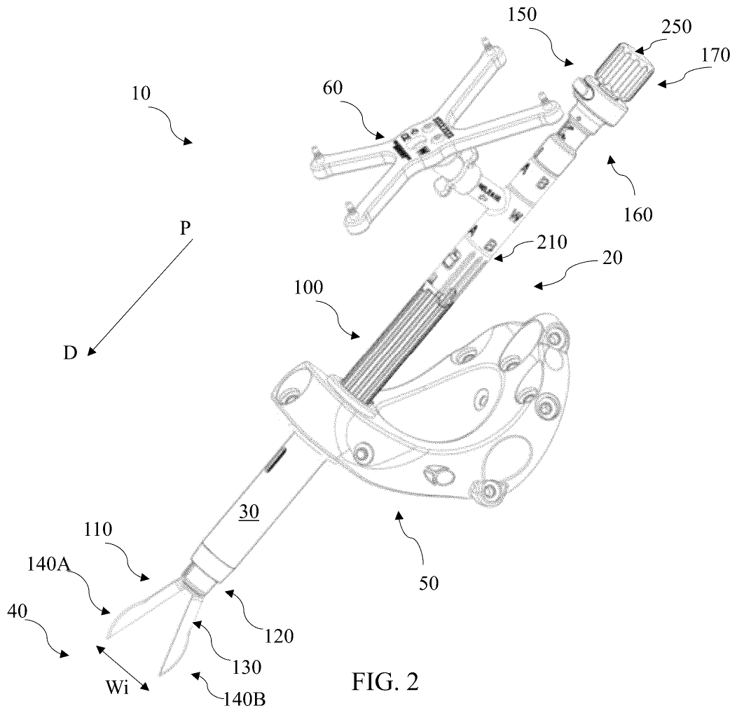

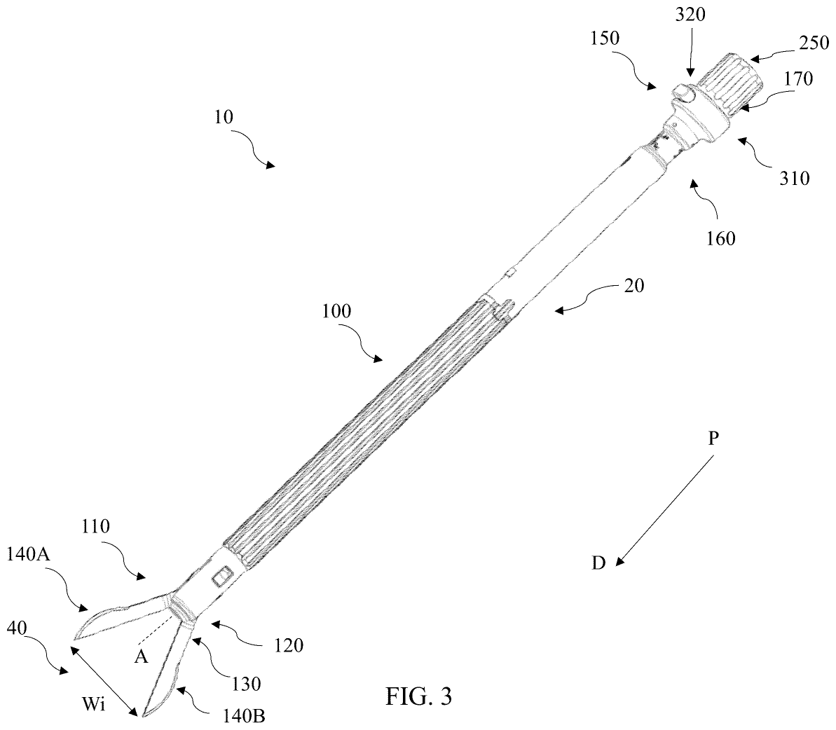

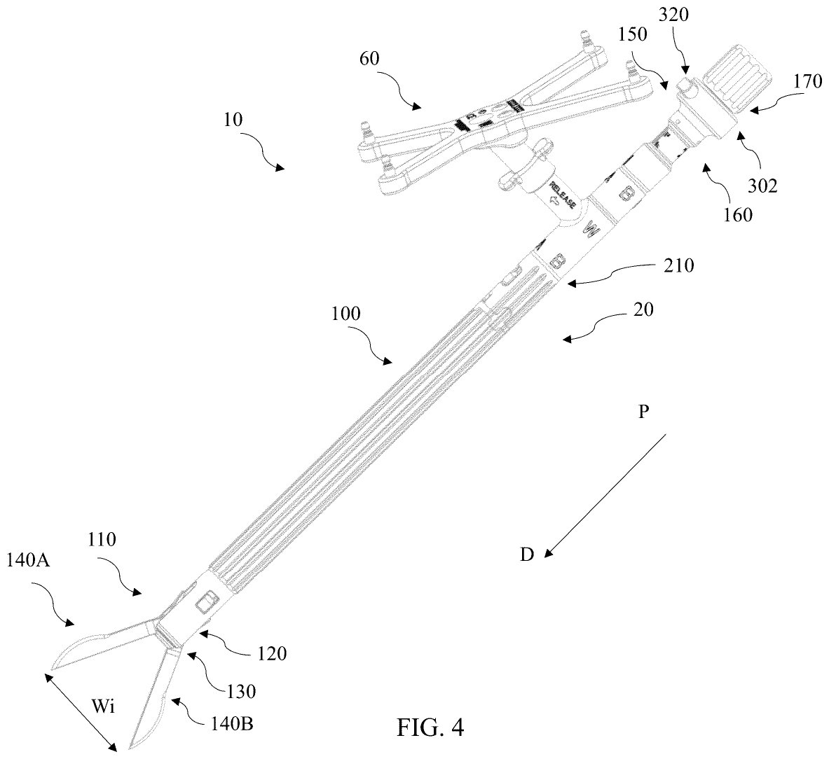

FIGS. 1 and 2 show the scalpel device 20 loaded in the guide tube 30, e.g., axially loaded from along the proximal (P) to distal (D) direction. FIG. 3 shows the scalpel device 20 removed from the guide tube 30. The scalpel device 20 can include a body 100 sized to translate within the guide tube 30, and an expandable cutting tool 110 coupled with a distal end 120 of the body 100. The expandable cutting tool 110 can include a cutting assembly 130 such as a scalpel blade 140 or a set of scalpel blades 140 including a plurality of blades 140 (two blades 140A, 140B shown in this example). FIG. 1 shows the blades 140 in a first position (e.g., retracted, or base position), and FIGS. 2 and 3 show the blades in a second position (e.g., extended, or separated position). FIG. 4 shows the additional, optional navigator 60 coupled with the scalpel device 20 according to certain implementations. In various implementations, the cutting tool 110 is sized to fit within the guide tube 30 when retracted, and expand radially beyond the guide tube 30 when extended.

In some cases, the blades 140 of the cutting tool 110 are configured to have an equal or greater outer dimension (e.g., as measured perpendicularly to the P-D direction) than the guide tube 30, e.g. than the inner diameter of the guide tube 30 when extended. When retracted, the blades 140 may collectively have a lesser outer dimension than the guide tube 30, e.g., the inner diameter of the guide tube 30, to enable the scalpel device 20 to be axially translated through the guide tube 30, for loading, unloading, and/or axial adjustment. In various implementations, blades 140 include a scalpel blade, and adjustment of the scalpel blade(s) includes at least one of: changing an angle of the scalpel blade relative to an axis (A) of the body 100 (labeled on FIGS. 1 and 3), translating the scalpel blade from the body 100, or retracting the scalpel blade within the body 100.

With continuing reference to FIGS. 1-4, the scalpel device 20 can also include an actuator 150 coupled with the proximal end 160 of the body 100. The actuator 150 can include a control feature 170 according to various implementations. The actuator 150 enables adjustment of the cutting assembly 130 according to an incision width (FIG. 2, Wi) selected from a set of incision widths defined by the control feature 170. FIGS. 1 and 2 illustrate the control feature 170 in distinct positions to control adjustment of the incision width (Wi) of the cutting assembly 130. Adjusting the incision width (Wi) of the cutting assembly 130 may result in a change in angle of each blade 140 relative to the central axis (A), e.g., a change from default position in FIG. 1 and an actuated position in FIG. 2.

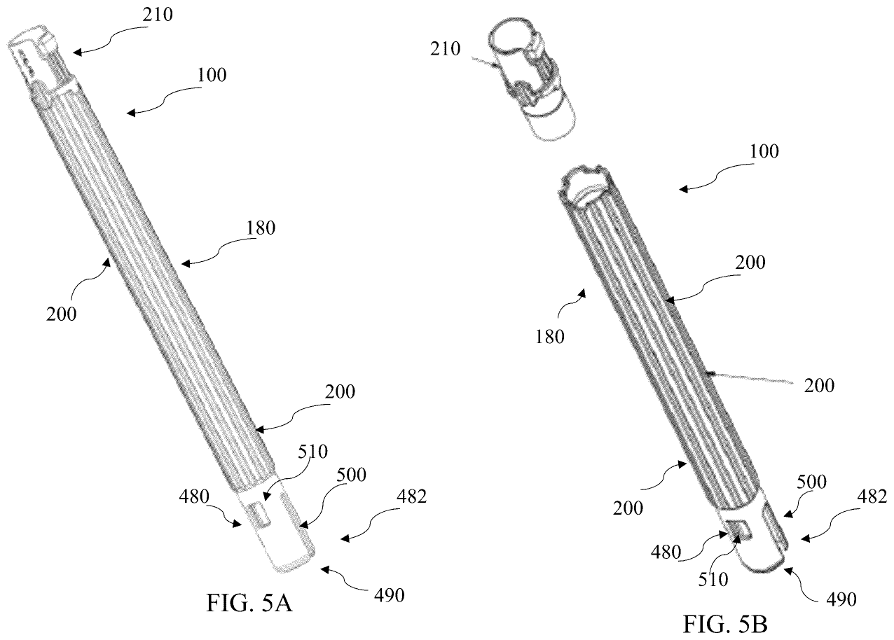

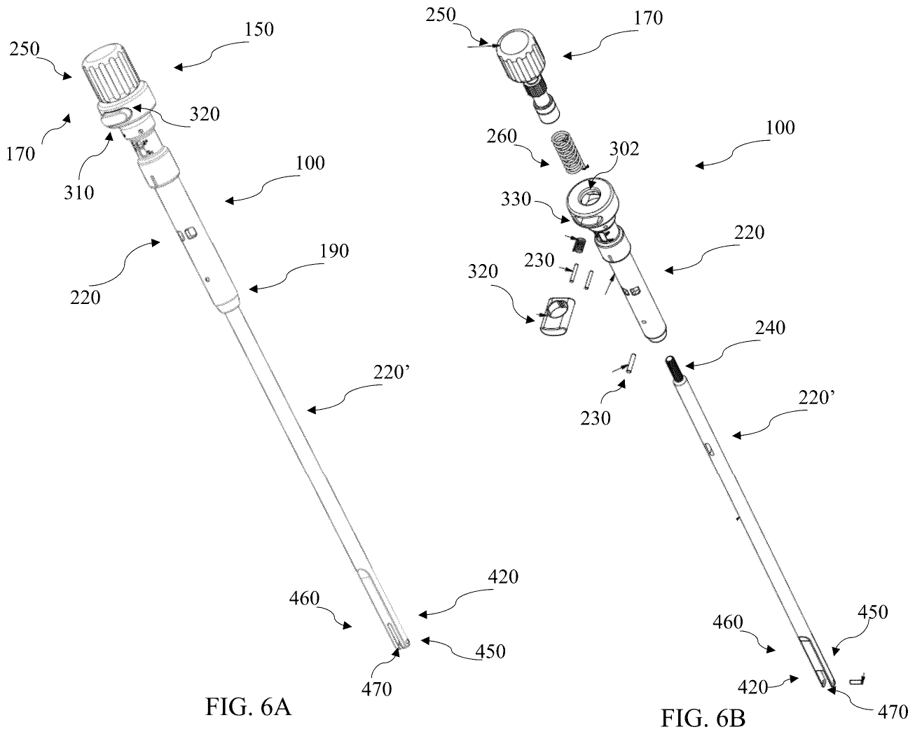

In certain cases, as illustrated in assembled views of the scalpel device 20 in FIGS. 3 and 4, and in the partially disassembled views of scalpel 20 in FIGS. 5A-6B, the body 100 can include an outer shaft 180 and an inner shaft 190 within the outer shaft 180. In certain examples, the inner shaft 190 sits within the outer shaft 180 and is approximately axially aligned with the outer shaft 180. In certain cases, the outer shaft 180 includes a set of external, axially extending grooves 200 for aligning the scalpel device 20 with the guide tube 30. For example, the external grooves 200 can align with one or more detents such as one or more internal grooves or tabs in the guide tube 20, e.g., to align the rotational position of the scalpel device 20 relative to the distal-proximal (D-P) direction. In a particular example, the external grooves 200 aid in orienting the cutting assembly relative to the patient. For example, in a lumbar procedure, an operator (e.g., surgeon) may wish to make vertical incision(s) that are aligned with a direction of the patient's muscle. The external grooves 200 can allow the operator to position the body 100 in the guide tube 30 (e.g., by engaging mating features in the guide tube 30) such that the blades 140 are aligned with the direction of the patient's muscle. In particular implementations, the outer shaft 180 includes an adapter cap 210 (e.g., at a proximal end thereof) that aids in connecting with a navigator 60 in certain implementations.

In certain cases, the inner shaft 190 is coupled with the actuator 150, which in some cases, includes a handle, dial, or other manually adaptable actuation mechanism. In particular cases, the inner shaft 190 has a plurality of segments 220 which can be connected via one or more couplers 230, e.g., pins, screws, etc. In certain cases, the segments 220 are coupled via complementary threaded connections 240. In certain implementations, as described herein, the actuator 150 includes a push-to-actuate and/or rotate-to-actuate feature. In one example such as illustrated in FIG. 6B, the actuator 150 can include a handle 250 that is coupled with an axially aligned spring 260 for enabling spring-loaded actuation of the cutting assembly 130. In a particular implementation, the spring 260 applies static pressure on the handle 250 in a resting or default position, keeping the blades 140 in a closed or retracted position such as illustrated in FIG. 1. In particular implementations, the force from spring 260 can be overcome by a push-to-actuate or rotate-to-actuate movement at handle 250, thereby transferring displacement of the actuator 150 to the inner shaft, in particular, to the distal segment 220′. The force from spring 260 can smooth the transition between the retracted (default) and extended positions, enabling the user to precisely locate a desired incision width.

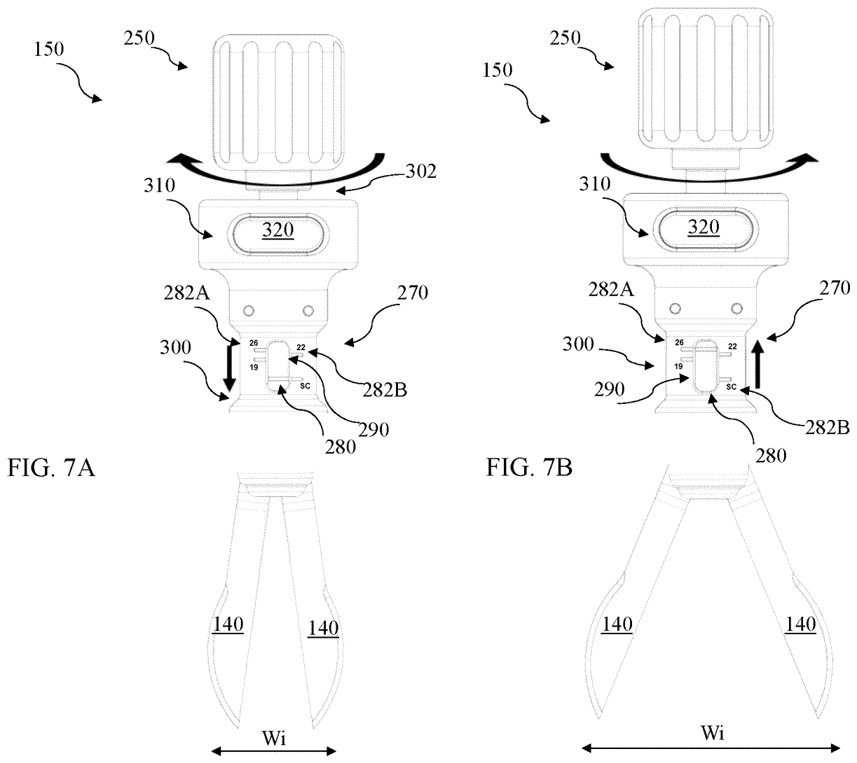

FIGS. 7A and 7B illustrate schematic views of an actuator 150 and corresponding blades 140 in a cutting assembly 130 in two distinct positions as controlled by the actuator 150. These views exclude the body 100 of the scalpel device 20 to provide greater detail about the actuator 150 and corresponding cutting assembly 130. In this example, the actuator 150 is shown including a visual indicator 270 of one or more incision widths available for selection. In a particular example, the visual indicator 270 includes a plurality of incision width indicators that can be selected by the user, e.g., with push-to-actuate and/or rotate-to actuate functions, e.g., by pushing and/or rotating handle 250. In certain cases, the visual indicator 270 can include an interface 280 that enables the user to preselect the desired incision width (Wi) from a range of incision widths ((Wi)s), e.g., a slider, a dial, a peg-based system, a tab-based system, a click-to-set or push-to-set mechanism, etc., In additional cases, the visual indicator 270 adjusts the displayed incision width at the interface 280 based on adjustment of the actuator 150, e.g., pushing and/or rotating the actuator 150. In certain examples, as the user engages with the actuator 150, the blades 140 in the cutting assembly are adjusted along with the visual indicator 270 corresponding with the adjusted incision width (Wi). In certain cases, the actuator 150 includes at least one of a multi-directional or multi-modal actuator for adjusting the incision width (Wi) of the blades 140. For example, the handle 250 can be rotated in both a clockwise direction (FIG. 7A) and a counterclockwise direction (FIG. 7B) to adjust the incision width (Wi) of the blades 140. FIGS. 7A and 7B illustrate adjustments made via handle 250 from a default position where the incision width (Wi) is approximately zero (e.g., blades 140 are closed, such as in FIG. 1). FIG. 7A shows a first actuation including compression (pressing) and clockwise rotation of the handle 250 to a first incision width (Wi) that is associated with a first procedure, e.g., a pedicle screw incision. In these cases, as the handle 250 is actuated, the visual indicator 270 is updated to align an indicator (e.g., line, marker, etc.) with a scale or range of incision widths (Wi). FIG. 7B shows a second actuation including counter-clockwise rotation of the handle 250 to a second incision width (Wi) that is associated with a second procedure, e.g., a port incision such as a 26 millimeter (mm) port incision. In this case, as the handle 250 is actuated, the visual indicator 270 is updated to align an indicator (e.g., line, marker, etc.) with a scale or range of incision widths (Wi).

In particular implementations, the visual indicator 270 includes a set of markers 282 configured to provide visual feedback on an amount of rotation of the handle 250. In certain cases, the markers 282 include color-coded or location-differentiating indicators that indicate an amount the blades 140 are displaced from a baseline, or in certain cases, a resulting incision width (Wi) of the blades 140. For example, markers 282 can include distinct columns 282A, 282B, etc., that are arranged next to a window 290 that illustrates incision widths (Wi) corresponding to the positions of the blades 140. In various implementations, the markers 282 are located on a fixed housing 300, such that as the actuator 150 is adjusted, e.g., handle 250 is rotated or translated, a user can view the state of the blades 140 via the markers 282.

In certain implementations, e.g., as illustrated in FIGS. 1-3, and more particularly in FIG. 6A, the actuator 150 can also include a control feature 170 including a lock 302 (FIGS. 6B, 7A) for fixing a position of the cutting assembly 130, e.g., an axial position of the cutting assembly 130 and/or an angular position of the blades 140. In particular cases, the lock 302 includes a threaded coupler as illustrated in FIG. 6B, which can fix a position of the cutting assembly 130, e.g., during a procedure. In another implementation, the lock can include one or more notches for engaging the actuator 150 to limit movement of the internal shaft 190 and consequently, the cutting assembly 130.

In a particular example, the control feature 170 includes a limiter 310 that limits the push-to-actuate or rotate-to-actuate feature. Returning to FIGS. 6A-7B, in certain cases, the limiter 310 includes a tab or protrusion 320 that mates with a slot 330 in the housing 300. In some of these cases, when locked, the tab or protrusion 320 protrudes from the slot 330 and maintains the position of the actuator 150 (and consequently, the blades 140). In certain cases, the limiter 310 can include a release mechanism, such as a push-to-release mechanism, for releasing the actuator 150. In these cases, in response to a user pushing the protrusion 320, the spring 260 causes actuator 150 to revert to its default position, e.g., a retracted or base position such that blades 140 are closed. In further implementations, the limiter 310 can include a notch, a stop, a spring-loaded mechanism, and/or threads that limit the push-to-actuate or rotate-to-actuate feature.

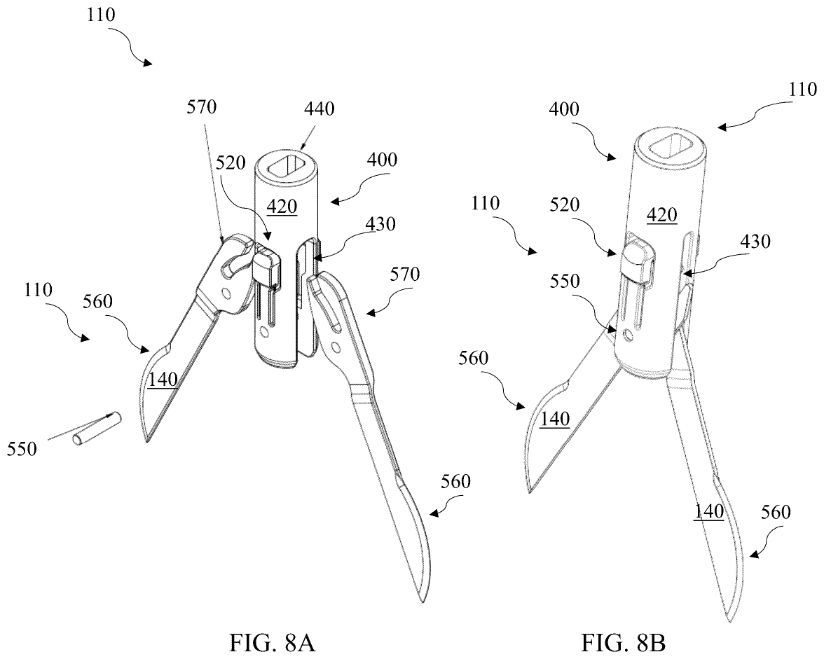

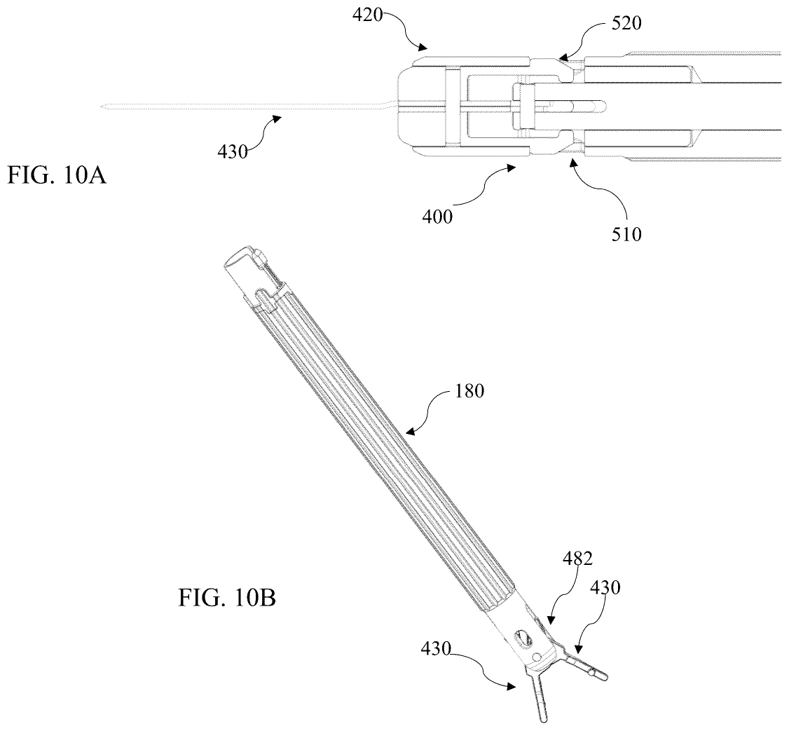

Turning to FIGS. 8A and 8B, and with continuing reference to FIGS. 5A through 6B, in various implementations, the expandable cutting tool 110 includes a cartridge 400 that is removably coupled with a distal end 120 of the body 100, e.g., a distal end 420 of the inner shaft 190. In various implementations, the cartridge 400 includes a main body 420, a set of blade mounts 430, and one or more blades 140 coupled with the corresponding blade mounts 430. In certain cases, the cartridge 400 includes a slot 440 that is configured to mate with a complementary coupler 450 on the distal end 460 of the inner shaft 190 (FIGS. 6A-6B). In certain cases, the coupler 450 includes at least one mating feature 470 such as one or more prongs that are configured to mate with a mount in the cartridge 400. In certain cases, the distal end 460 of the inner shaft 190 fits in the slot 440 and is configured to translate within the slot 440 to actuate movement of blades 140. In particular cases, the outer shaft 180 includes at least one retaining feature 480 proximate its distal end 482 for coupling with the cartridge 400 (FIGS. 5A-5B). In certain cases, the cartridge 400 is configured to slide axially into and out of a sheath 490 at the distal end 482 of the outer shaft 180. In one example, the sheath 490 includes at least one slot 500 extending in a proximal direction from the distal end 482 and a set of apertures (or, slots) 510 for engaging a set of tabs (or, protrusions) 520 on the cartridge 400. In certain examples, the slot 500 is open at the distal end 482 of the outer shaft 180. In certain cases, the cartridge 400 can be inserted axially by aligning the tab(s) 520 with slot(s) 500, and rotating the cartridge 400 until the tab(s) 520 engage the slot(s) 510. The reverse of this process can be performed to remove the cartridge 400 from the sheath 490. In some cases, the tabs 520 extend radially beyond an outer surface of the main body 420, and in particular cases, are formed of a compliant material or are otherwise at least partially deformable to enable a snap-to-fit connection with the outer shaft 180. In various implementations, the cartridge 400 is retained in the outer shaft 180 by the connection between tabs 520 and slots 510. This coupling is further illustrated in FIG. 10A.

In particular implementations, the cartridge 400 is configured to be removably coupled from the body 100, and in certain cases, can be separately packed relative to the body 100. For example, the cartridge 400 and/or the blades 140 can be separately packed and/or sterilized to enable those components to be modularly connected with the body 100. In some cases, the cartridge 400 and/or blades 140 are disposable or configured for one-time use.

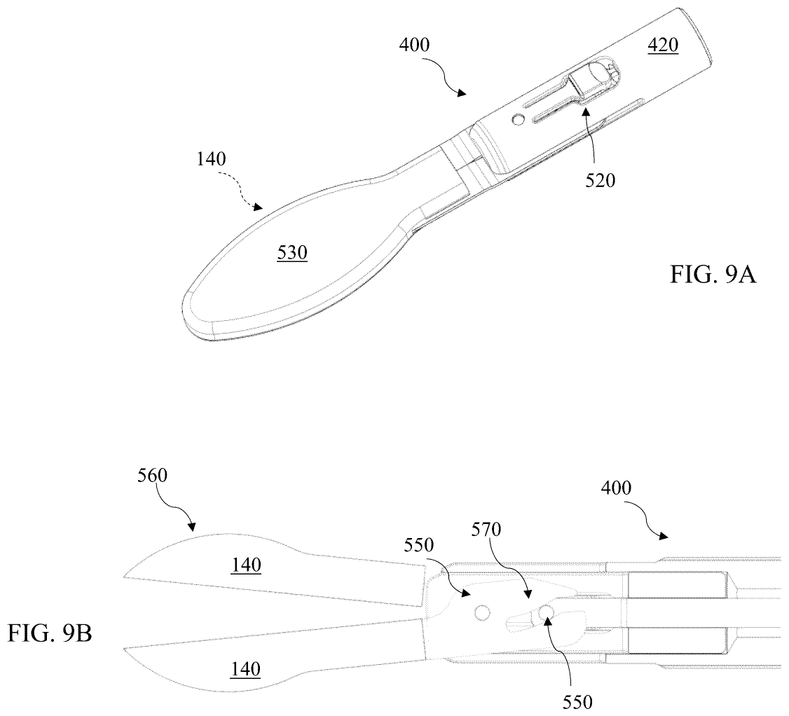

In certain cases, as illustrated in FIG. 9A, a cover 530 can be configured to fit over the blades 140 to enable safe and/or secure transport and mounting/dismounting of the blades 140 and/or cartridge 400 from the body 100.

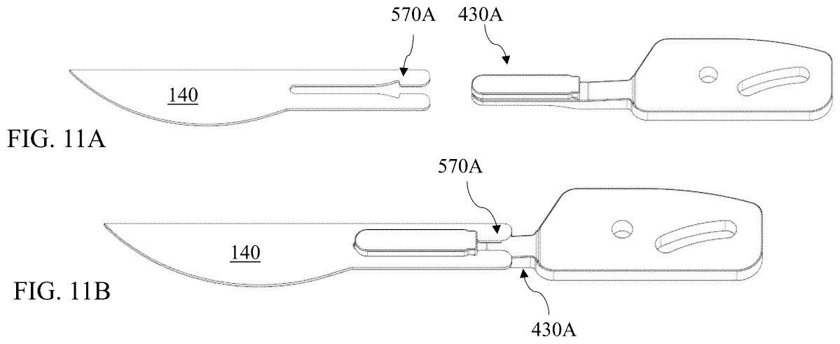

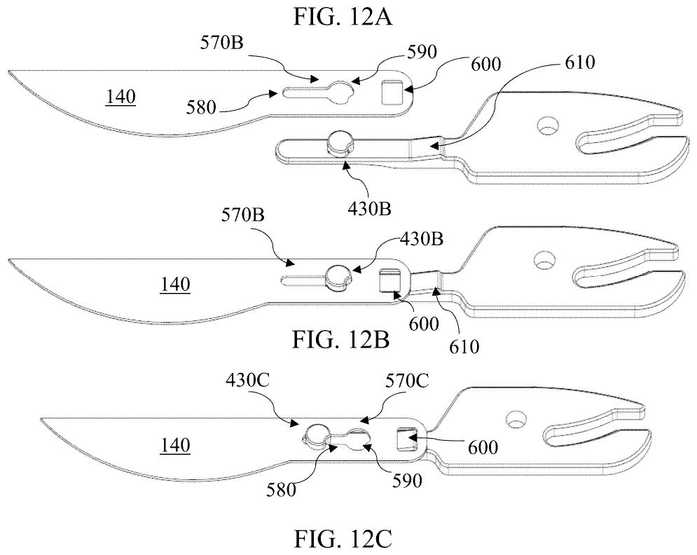

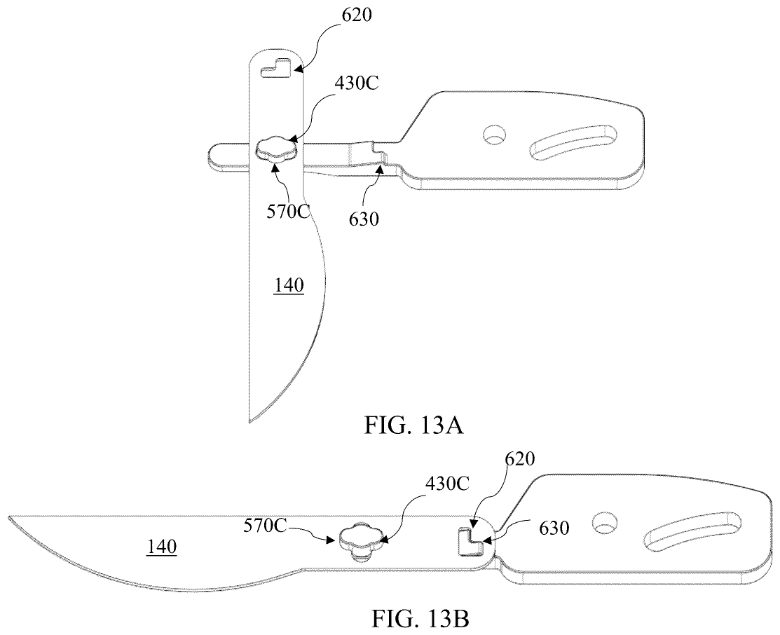

With continuing reference to FIGS. 8A and 8B, and with additional reference to FIG. 9B, in particular examples, the blade mounts 430 can include pins and/or additional securing features 550 that can be removable to enable loading and unloading of blades 140 from the body 420. In certain cases, the blades 140 are pivotably coupled with the body 420 and/or each other, such that adjustment of an actuator (e.g., translation of inner shaft 190) causes the blades 140 to pivot inward or outward relative to a default position). As noted herein and illustrated in FIGS. 8A, 8B, and 9B, each blade 140 can include a cutting edge 560 and a coupler 570 for connecting with the blade mounts 430. In certain cases, the coupler 570 can include a slot for mounting to an attachment tip. In some examples, the coupler 570, e.g., slot, can have an orientation feature such as a snap-to-fit or twist-to-fit connector that enables coupling in only one direction. FIGS. 11A-13B illustrate examples of blades 140 with distinct couplers 570A, 570B, 570C for connecting with distinct types of blade mounts 430A, 430B, 430C, respectively. For example, FIGS. 11A and 11B illustrate a coupler 570A with a spring-style slot that enables a snap-to-fit or press-to-fit coupling with blade mount 430A on a blade 140. FIGS. 12A-12C illustrate a coupler 570B such as, e.g., an elongated slot 580 with a keyhole opening 590 in blade 140 adapted to receive a complementary-shaped protrusion or knob disposed on the blade mount 430A. In this case, a secondary coupler 600 on the blade 140 such as a slot or mating feature is shown for coupling with a complementary coupler 610 on the mount 430B. FIGS. 13A-13B illustrate a coupler 570C such as a directional protrusion or knob that enables a keyed fit with blade mount 430C that has an orientation feature, e.g., a twist-to-secure/twist-to-remove fit. In this case, a secondary coupler 620 on a blade 140 such as a slot or mating feature is shown for coupling with a complementary coupler 630 on the mount 430C.

In certain additional implementations, such as illustrated in FIG. 10B, blade mounts 430 can be located on the outer shaft 180, e.g., such that the blades 140 are directly mounted to the outer shaft 180 via the blade mounts 430. The example blade mounts 430A-430C illustrated in FIGS. 11A-13B are compatible with these configurations, for example, where blade mounts 430A-430C are connected with and extend directly from the distal end 482 of the outer shaft 180 (FIG. 10B). In certain of these cases, the blades 140 are configured to be installed and/or removed separately from the mounts 430, e.g., to enable separate sterilization, packaging, or delivery of the blades 140. In some of these example implementations, the blades 140 are disposable and/or configured for one-time use.

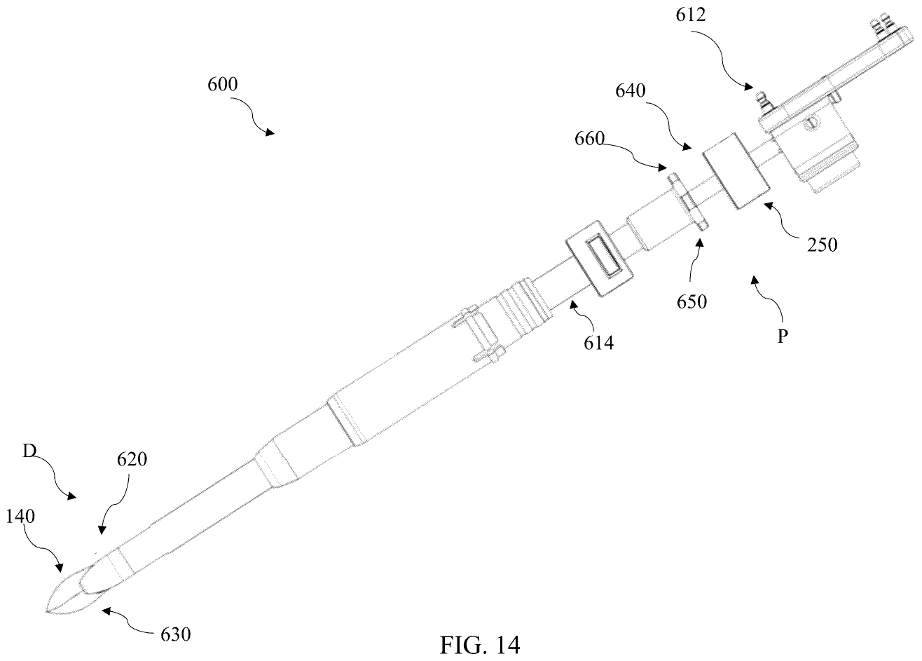

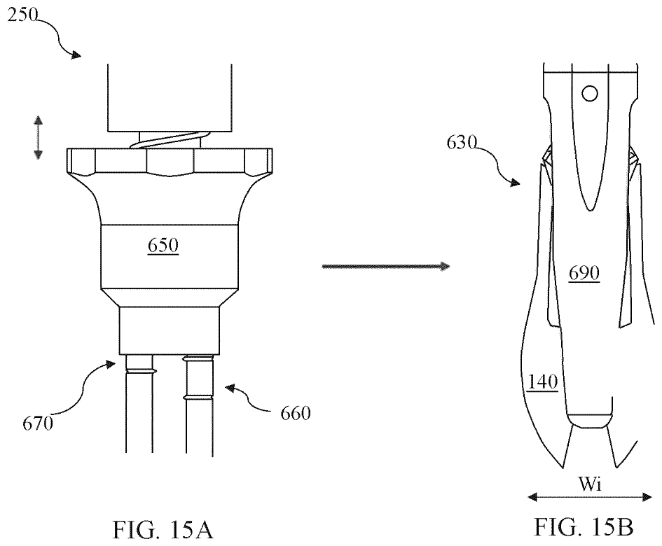

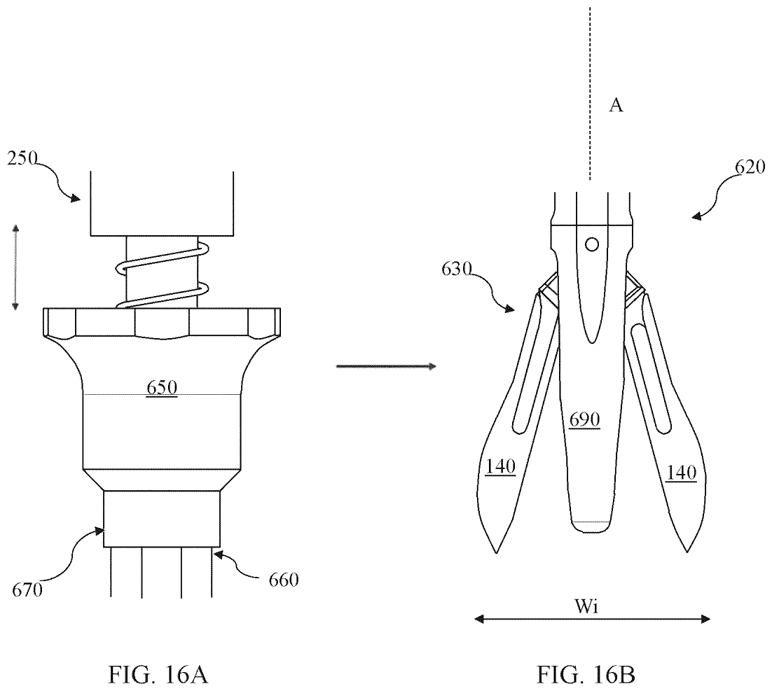

FIG. 14 illustrates another implementation of a scalpel device 600 including a plurality of similar features to scalpel device 20. In this implementation, an optional navigation device 612 is coupled with a proximal end (P) of the scalpel device and configured to aid in navigation of the scalpel device 600 with a guide tube 30. In particular cases, the scalpel device 600 includes a body 614, an expandable cutting tool 620 including a cutting assembly 630 coupled with a distal end (D) of the body 614, and an actuator 640 including a control feature 650 coupled with a proximal end (P) of the body. In certain cases, the actuator 640 includes a spring-loaded actuator, and includes various features similar to the actuator 150 in FIGS. 1-7B. In certain examples, the actuator 640 enables adjustment of the cutting assembly 630 according to an incision width selected from a set of incision widths. In particular cases, the cutting assembly 630 is adjusted by translation triggered by the actuator 640, e.g., a push-to-adjust and/or twist-to-adjust motion at the actuator 640. In certain cases, the control feature 650 includes a limiter 660 such as a threaded mechanism 670 for limiting translation of the control feature 650, and consequently, the expansion or contraction of blades 140, as illustrated in FIGS. 15A-B and 16A-B.

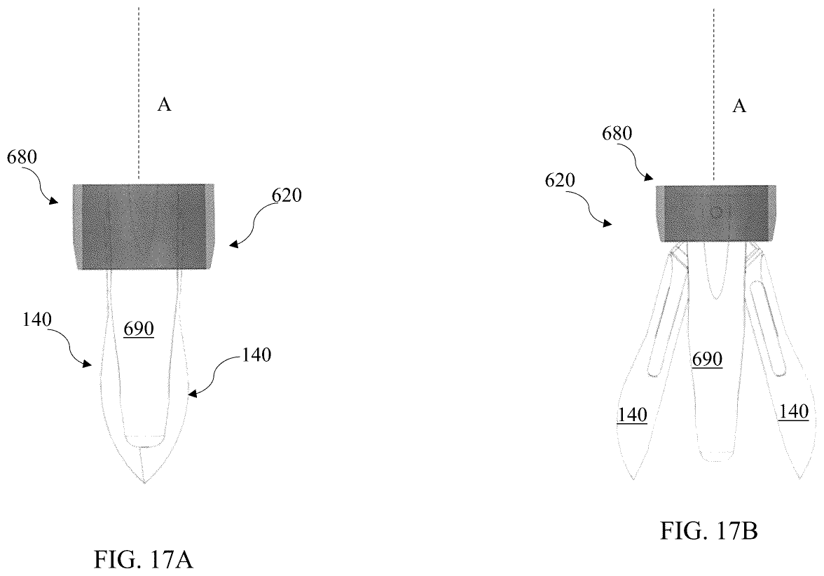

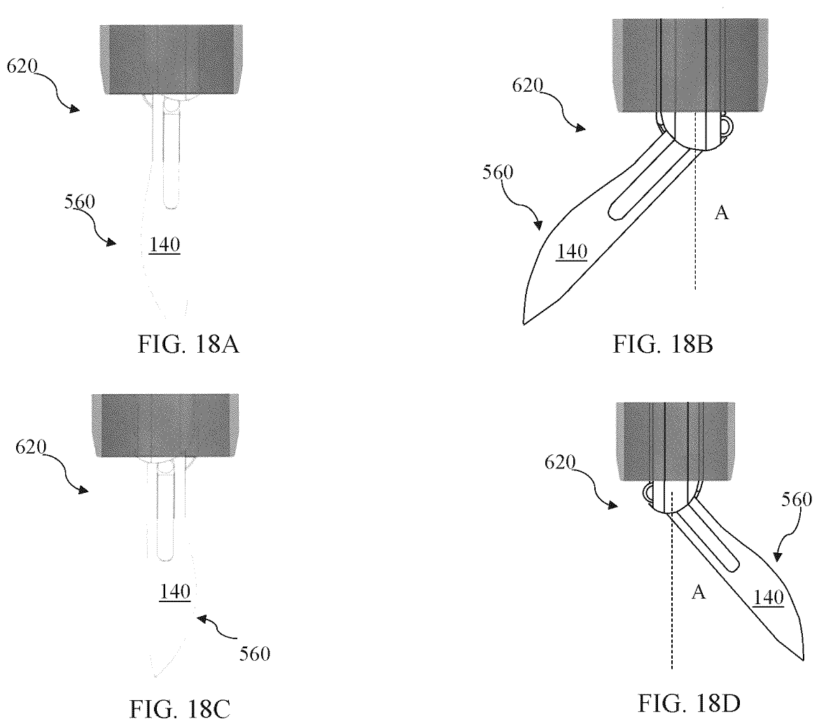

FIGS. 17A-B illustrate additional implementations in which the cutting tool 620 includes a mount 680 for coupling with a distal end (D) of the body 614 (FIG. 14), including a set of two blades 140 configured to expand and contract relative to a guide member 690. As noted according to various implementations herein, the blades 140 can be pivotably coupled to one another in some cases. In other cases, the blades 140 are pivotably coupled to the body, and are independently adjusted/adjustable. FIGS. 18A-18D illustrate another implementation in which the cutting tool 620 includes a single blade 140 configured to be expanded/retracted in multiple directions (e.g., FIGS. 18B, 18D), e.g., changing the blade angle relative to the axis (A) of the body, and/or oriented in distinct directions (FIGS. 18A, 18C) via control from the actuator 640. In certain of these implementations, the actuator 640 can be rotated at least 180 degrees to cause the blade 140 to change orientation relative to axis (A), e.g., as shown in FIGS. 18A and 18C. In these cases, change in angle can be controlled by a push-to-actuate or rotate-to-actuate function as described herein. That is, translation of the body 614 causes the single blade 140 to change angle relative to axis (A) as described in multi-blade configurations herein. In certain of these examples, orientation of the blade 140 can be adjusted prior to translation of the body 614.

In various implementations, the scalpel device(s) 20, 600 can be incorporated in any number of surgical management systems, such as robotic surgical management systems. One example of such a surgical management system is described in U.S. Pat. No. 11,083,527 (“Systems and methods for assisted surgical navigation”), issued Aug. 21, 2021, which is incorporated by reference herein. Further, the scalpel device(s) 20, 600 can be beneficially deployed with any number of guide devices and/or transport devices in a surgical management system, e.g., as disclosed in U.S. Pat. No. 10,136,927 (issued Nov. 27, 2018), US Patent Application Publication Nos. 2020/0297393 (U.S. application Ser. No. 16/898,713) and 2021/0085485 (U.S. application Ser. No. 16/995,602), each of which is incorporated by reference in its entirety.

It is understood that any of the disclosed scalpel devices (e.g., scalpel device 20, scalpel device 600) can be utilized as part of a surgical robot system, for example, a robot having a robot base, a robot arm coupled to the robot base, and a transport device such as an end-effector coupled to the robot arm. In various implementations, a transport device, e.g., an end effector such as end effector 50 in FIG. 1 can enable receiving and/or guiding of the scalpel device to aid in a procedure such as a surgical procedure.

In additional implementations, transport devices such as end effectors can also include or be configured to receive a handpiece such as a power screwdriver, and can be configured to communicate with other devices and/or systems in a surgical management system.

In some cases, sensors such as optical sensors, pressure and/or torque sensors, etc., are integrated in or complement the surgical robot system and provide sensor readings during a surgical procedure. These readings are converted to inputs for recordation and/or output (via visual, audible, etc. feedback), e.g., by a surgical management system such as those described herein. In further implementations, components of the surgical management system such as an electronics package are located at the transport devices and/or robot. In other cases, an electronics package is remote from those devices. As noted herein, one or more of the beneficial features of the disclosure can be applied to transport devices not specifically depicted or described. For example, rod reducers, removal tools, etc., can benefit from the disclosed approaches of the various implementations.

Further, as noted herein, scalpel devices 20, 600 can include or otherwise be coupled with electronics, which can be configured to communicate (e.g., wirelessly and/or hard-wired connection) with a remote surgical management system. In some cases, the electronics include or are coupled with sensors, a power source, and signal conditioning electronics such as an interface circuit to process and output a signal. In certain cases, the interface circuit includes a signal processor such as a digital signal processor (DSP), a logic engine to filter/condition the signal, and a controller to control onboard functions such as displays and transmission of signals to external components such as an external receiver. Additionally, the sensors can provide feedback to a medical professional (e.g., surgeon) during and/or after a procedure via the surgical management system.

In particular implementations, the surgical management system includes a feedback system that has a controller (e.g., one or more microcontrollers) with least one processing unit, or processor (PU) (such as one or more microprocessors) coupled with or containing a memory (e.g., including one or more storage components such as memory chips and/or chipsets). The memory stores instructions (e.g., surgical calculation and/or feedback instructions) which when executed by the PU(s) cause the PU to: compare data from one or more sensors with corresponding surgical threshold(s), and provide feedback about the data comparison(s).

Devices such as scalpel devices, transport devices, end effectors, and/or other handpieces described herein are described as including communication devices and related electronics. These communications device(s) can include one or more transmitters and/or receivers (e.g., wireless and/or hard-wired transmitters/receivers). In various implementations, the communication devices are configured for a plurality of communication protocols, e.g., wireless protocols such as Wi-Fi, Bluetooth, BLE, Zigbee, etc., as well as radio communication and intercom communications, and/or a hardwired connection (e.g., fiber optic connection).

The devices described herein can also be coupled with a navigation system in order to provide navigation information about a position of one or more instruments. For example, the navigation system can include an optical tracking system such as a camera or laser-based tracking system, a Global Positioning System (GPS), an inertial measurement unit (IMU), etc. In certain cases, the navigation system is configured to determine a distance moved by the instrument when the instrument changes position, which the navigation system communicates to the feedback system. One or more components of a navigation system can be located within or otherwise integrated with a housing that is mounted to or otherwise coupled with one or more of the transport device(s). The feedback system can also be configured to provide post-operative data and analysis of a procedure and/or device usage, e.g., to enhance future procedures and/or diagnose inefficiencies in a past procedure. In certain implementations, the feedback system is configured to update transport device instructions based on identified inefficiencies or errors in transport device sequencing and/or device usage during a given procedure. In particular implementations, the feedback system includes a logic engine configured to modify transport device instructions iteratively, e.g., on a procedure-by-procedure basis.

As noted herein, the scalpel devices and related systems disclosed according to various implementations provide numerous benefits relative to conventional devices and systems. For example, the scalpel device enables an operator such as a surgeon to control an incision width of a cutting assembly from a set of incision widths, for example, using an actuator at a proximal end of the device body. In certain cases, the scalpel enables fixing the position of the cutting assembly. In various implementations, the cutting assembly enables adjustment of an angle of a scalpel blade relative to the body, translation of the scalpel blade from the body, and/or retraction of the scalpel blade within the body. These devices and systems can enhance surgical outcomes for patients, and increase efficiency in surgical procedures.

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultrasonic welding, bonding). In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

While inventive features described herein have been described in terms of preferred embodiments for achieving the objectives, it will be appreciated by those skilled in the art that variations may be accomplished in view of these teachings without deviating from the spirit or scope of the invention. Also, while this invention has been described according to a preferred use in spinal applications, it will be appreciated that it may be applied to various other uses desiring surgical fixation, for example, the fixation of long bones.

A number of implementations have been described. Nevertheless, it will be understood that additional modifications may be made without departing from the scope of the inventive concepts described herein, and, accordingly, other implementations are within the scope of the following claims.

We claim:

1. A scalpel device comprising:
a body sized to translate within a guide tube for delivery to a patient;
an expandable cutting tool coupled with a distal end of the body, the expandable cutting tool including a cutting assembly; and
an actuator including a control feature coupled with a proximal end of the body, wherein the actuator enables adjustment of the cutting assembly according to an incision width selected from a set of incision widths defined by the control feature.

2. The scalpel device of claim 1, wherein the control feature includes a lock for fixing a position of the cutting assembly.

3. The scalpel device of claim 1, wherein the cutting assembly includes a scalpel blade, and wherein adjustment of the scalpel blade includes at least one of: changing an angle of the scalpel blade relative to an axis of the body, translating the scalpel blade from the body, or retracting the scalpel blade within the body.

4. The scalpel device of claim 4, wherein the scalpel blade includes a plurality of scalpel blades pivotably coupled to one another.

5. The scalpel device of claim 1, wherein the guide tube includes a dilator.

6. The scalpel device of claim 1, wherein the body includes an outer shaft and an inner shaft within the outer shaft.

7. The scalpel device of claim 6, wherein the outer shaft includes a set of external grooves for aligning the scalpel device with the guide tube, wherein the set of external grooves aids in orienting the cutting assembly relative to the patient.

8. The scalpel device of claim 1, wherein the actuator is coupled with a proximal end of the body.

9. The scalpel device of claim 8, wherein the expandable cutting tool includes a cartridge that is removably coupled with a distal end of the body, wherein the cartridge includes:
a main body,
a set of blade mounts, and
a set of blades connected with the set of blade mounts.

10. The scalpel device of claim 9, wherein each blade has a cutting edge and a coupler for connecting with the set of blade mounts.

11. The scalpel device of claim 8, wherein the expandable cutting tool includes a set of blade mounts coupled with a distal end of the body, wherein the set of blade mounts enable detachable coupling of at least one blade to the body.

12. The scalpel device of claim 1, wherein the actuator includes at least one of a push-to-actuate or a rotate-to-actuate feature.

13. The scalpel device of claim 12, wherein the control feature includes a limiter that limits the push-to-actuate or rotate-to-actuate feature.

14. The scalpel device of claim 13, wherein the limiter includes a release mechanism for releasing the actuator.

15. The scalpel device of claim 14, wherein actuating the release mechanism causes the actuator to revert to a default position.

16. The scalpel device of claim 1, wherein the actuator includes a visual indicator of the incision width.

17. The scalpel device of claim 16, wherein adjustment of the actuator causes a change in the visual indicator of the set of incision widths.

18. The scalpel device of claim 1, wherein the expandable cutting tool is sized to fit within the guide body when retracted and expand radially beyond the guide body when extended.

19. A surgical robot system comprising the scalpel device of claim 1.

20. The surgical robot system of claim 19, further comprising:
a robot having a robot base, a robot arm coupled to the robot base, and an end-effector coupled to the robot arm, the end effector including the guide tube for receiving the body of the scalpel device.
